(19) United States
(12) Patent Application Publication
(10) Pub. No.: US 2016/0067108 A1
(43) Pub. Date: Mar. 10, 2016

(54) SOLE STIMULATION SOCK

(71) Applicant: OKAMOTO CORPORATION, Kitakatsuragi-gun (JP)

(72) Inventors: Tsuneo KAWAGOE, Kitakatsuragi-gun (JP); Ichiro SHINGA, Kitakatsuragi-gun (JP); Hidekazu TANAKA, Kashihara-shi (JP); Akira TANIGUCHI, Kashihara-shi (JP); Yasuhito TANAKA, Kashihara-shi (JP)

(21) Appl. No.: 14/844,080

(22) Filed: Sep. 3, 2015

(30) Foreign Application Priority Data
Sep. 5, 2014 (JP) 2014-181620

(51) Int. Cl. A61F 13/06; D04B 1/26; A41B 11/00

(52) U.S. Cl. CPC A61F 13/064; A41B 11/004; D04B 1/26

(57) ABSTRACT

There is provided a sole stimulation sock, in which an inner side of a sole portion between a heel portion and a toe portion is formed with projected portions and recessed portions, in which a proportion of an area of the projected portions, with respect to the sum of the areas of the projected portions and the recessed portions, is 26% to 68%, in which the difference between a proportion of an area of the projected portions in any one division region of four division regions of the inner side of the sole portion and a proportion of an area of the projected portions in other division regions is less than or equal to 30%, in which the average value of the areas of the projected portions is 7 mm² to 650 mm², and in which the recessed portions are knitted by mesh knitting.

### CROSS REFERENCE TO RELATED APPLICATION

This application claims the benefit of Japanese Patent Application No. 2014-181620 filed on Sep. 5, 2014, all of which are incorporated herein by reference in their entirety.

### BACKGROUND OF THE INVENTION

1. Field of the Invention

The present invention relates to socks which stimulate the soles of a wearer, and particularly to socks for improving neuropathy by stimulating the soles of a wearer who has neuropathy in the feet due to diabetes or the like.

2. Related Background Art

For example, there is a circulatory disorder in terminal tissue as a complication of diabetes. Japanese Registered Utility Model No. 3098891 and Japanese Unexamined Patent Application Publication No. 2005-15949 disclose socks which are applied to a person with diabetes or a person who feels coldness in the toes, for example, a person with sensitivity to cold. The socks disclosed therein are double socks in which outer socks and inner socks that are continuously knitted are superimposed on each other, and the inner surface(s) of a toe portion and/or a bottom portion of at least the inner socks is/are formed on an irregular surface using a knitted texture. Accordingly, it is possible to create an air layer between the inner socks and the outer socks and it is also possible to create an air layer inside the recessed portions. Therefore, it is possible to achieve results such as heat insulation and heat retention and to improve moisture retaining properties and air permeability.

In addition, Japanese Unexamined Patent Application Publication No. 2008-279125 discloses socks in which an elastic member, which is formed of a rubber material and has elasticity, is brought into contact with a reflection region of the sole of the foot relating to internal organs which are specified by a sole reflection zone. Accordingly, it is possible to activate the circulation of blood of the sole of the foot and to improve a metabolic function of a specific internal organ by applying stimulation to the internal organ. Japanese Unexamined Patent Application Publication No. 2008-279125 discloses an arrangement position of an elastic member with respect to a patient with diabetes.

### SUMMARY OF THE INVENTION

For example, as complications of diabetes, there is neuropathy and infectious disease in a terminal tissue in addition to the circulatory disorder in a terminal tissue. If neuropathy develops in the feet of a person, it is difficult for the person to recognize external injuries in the feet and the person easily falls down. In general, it is recommended to wear socks for protecting the feet or keeping the feet warm. However, the development risk of infectious disease is increased due to the growth of bacteria caused by stuffiness. Furthermore, if neuropathy and infectious disease progress, necrosis occurs in the feet, and in some cases, the feet need to be amputated.

In some cases, manual mechanical stimulation is given to the feet of a patient with diabetes from a health professional (for example, medical practitioner and physical therapist) as part of foot care. The mechanical stimulation refers to physical stimulation such as contact-type stimulation and pressure-type stimulation, and is distinguished from thermal stimulation, optical stimulation, chemical stimulation, and the like. In addition, the manual mechanical stimulation refers to stimulation which is given to the feet of a patient (particularly, patient with neuropathy) with diabetes by a health professional using the fingertips, and is stimulation which is caused by applying a comparatively low amount of pressure a plurality of times with a comparatively light touch.

The present inventors have obtained knowledge that neuropathy is improved by such manual mechanical stimulation. The manual mechanical stimulation can be received from a health professional by going to the hospital, but is also desirably applied to the feet by himself/herself daily. However, applying the manual mechanical stimulation to the feet by himself/herself everyday requires a lot of time.

An object of the present invention is to provide a sole stimulation sock which can improve neuropathy in a foot portion by only being worn in daily life, and to suppress the development of infectious disease in the foot portion.

According to an aspect of the invention, there is provided a sole stimulation sock, in which an inner side of a sole portion in a cylindrical portion between a heel portion which covers the heel and a toe portion which covers the toes is formed with projected portions and recessed portions, in which the inner side of the sole portion, a proportion of an area of all of the projected portions, with respect to the sum of the area of all of the projected portions and an area of all of the recessed portions, is 26% to 68%, in which the difference between a proportion of an area of the projected portions in any one division region of four division regions and a proportion of an area of the projected portions in other division regions is less than or equal to 30%, the four division regions being obtained such that the inner side of the sole portion is equally divided into two regions in a right-left direction and a toe-heel direction, in which the average value of the areas of the projected portions is 7 mm² to 650 mm², and in which the recessed portions are knitted by mesh knitting.

According to the sole stimulation sock, the average value of the areas of the projected portions in the inner side of the sole portion is 7 mm² to 650 mm² which is a size equivalent to the ball of a finger, and the proportion of the area of all of the projected portions is 26% to 68% with respect to the sum of the area of all of the projected portions and the area of all of the recessed portions. Therefore, it is possible to reproduce stimulation which is equivalent to manual mechanical stimulation which is applied by a health professional (for example, medical practitioner and physical therapist). In addition, the difference between the proportion of the area of the projected portions in any one division region of the four division regions, which are obtained such that the inner side of the sole portion is equally divided into two regions in a right-left direction and a toe-heel direction, and the proportion of the area of the projected portions in other division regions is less than or equal to 30%. Therefore, the projected portions are sparsely scattered in the inner side of the sole portion without being deviated. Accordingly, it is possible to apply stimulation which is equivalent to manual mechanical stimulation which is applied by a health professional, to the sole by only being worn in daily life (for example, only by walking) and to improve neuropathy in a foot portion.

In addition, according to the sole stimulation sock, it is possible to secure air permeability since the recessed portions of the sole portion are knitted by mesh knitting. Accordingly, it is possible to suppress the growth of bacteria caused by stuffiness and to prevent the development of infectious disease of a foot portion.

The projected portions may be knitted by pile knitting. Accordingly, the projected portions have softness equivalent to the ball of a finger, and therefore, it is possible to further reproduce stimulation which is equivalent to manual mechanical stimulation which is applied by a health professional.

The projected portions may be formed of a resin, a synthetic resin, or synthetic rubber.

In addition, an instep portion in the cylindrical portion may be knitted by mesh knitting. Accordingly, it is possible to further secure air permeability. Therefore, it is possible to further suppress the growth of bacteria caused by stuffiness and to prevent the development of infectious disease of a foot portion.

In addition, the inner side of the heel portion may be knitted by pile knitting. Accordingly, it is possible to protect the heel of a wearer from weighted stimulation during, for example, grounding (landing) in a walking motion.

In addition, the inner side of the toe portion may be knitted by pile knitting. Accordingly, it is possible to protect the toes of a wearer from weighted stimulation during, for example, kicking in a walking motion.

In addition, the toe portion may have a divided structure which is divided into two or more regions which accommodate the toes of a wearer. Accordingly, it is possible to suppress the socks from twisting while the socks are worn.

In addition, a grafting portion between the toe portion and the cylindrical portion may be knitted by a linking seam or a front rosso seam. Accordingly, it is possible to protect the toes of a wearer from stimulation due to the grafting portion between the toe portion and the cylindrical portion.

In addition, in the sole stimulation sock, processing of suppressing the growth of Trichophyton may be performed. Accordingly, it is possible to suppress the development of tinea pedis.

According to the present invention, it is possible to improve neuropathy in a foot portion by only being worn in daily life (for example, by only walking), and to suppress the development of infectious disease in the foot portion.

### BRIEF DESCRIPTION OF THE DRAWINGS

FIG. 1 is a sole side plan view of an inner side (back side, skin side) of a sole stimulation sock (for the right foot) according to an embodiment of the present invention in a state where the sole stimulation sock is folded in a planar shape.

FIG. 2 is an instep side plan view of the inner side (back side, skin side) of the sole stimulation sock (for the right foot) according to the embodiment of the present invention in the state where the sole stimulation sock is folded in a planar shape.

FIG. 3A is a view showing a projected portion and a recessed portion of a sole portion of a sole stimulation sock according to the embodiment of the present invention and FIGS. 3B to 3J are views showing projected portions and recessed portions of sole portions of sole stimulation socks according to modification examples of the present invention.

FIG. 4A is a sole side plan view of an inner side (back side, skin side) of a sole stimulation sock (for the right foot) according to an example of the present invention and FIG. 4B is an instep side plan view of the inner side of the sole stimulation sock of the example.

FIG. 5 is an explanatory view for illustrating a perception examination method.

FIGS. 6A and 6B are views showing a perception examination result of the hallux balls (FIG. 6A) and heel portions (FIG. 6B) of sole portions of 9 examinees (with diabetes and neuropathy) before and after wearing a sole stimulation sock according to Example 1 of the present invention.

FIG. 7 is an explanatory view for illustrating a toe gripping force examination method.

FIG. 8 is a view showing toe gripping force examination results of 9 examinees (with diabetes and neuropathy) before and after wearing a sole stimulation sock according to Example 1 of the present invention.

FIG. 9 is a view showing toe gripping force examination results of 5 examinees (with diabetes but without neuropathy) before and after wearing a sole stimulation sock according to Example 1 of the present invention.

### DESCRIPTION OF THE PREFERRED EMBODIMENT

Hereinafter, a preferred embodiment of the present invention will be described in detail with reference to the drawings. The same reference numerals will be given to the same or corresponding members in each of the drawings.

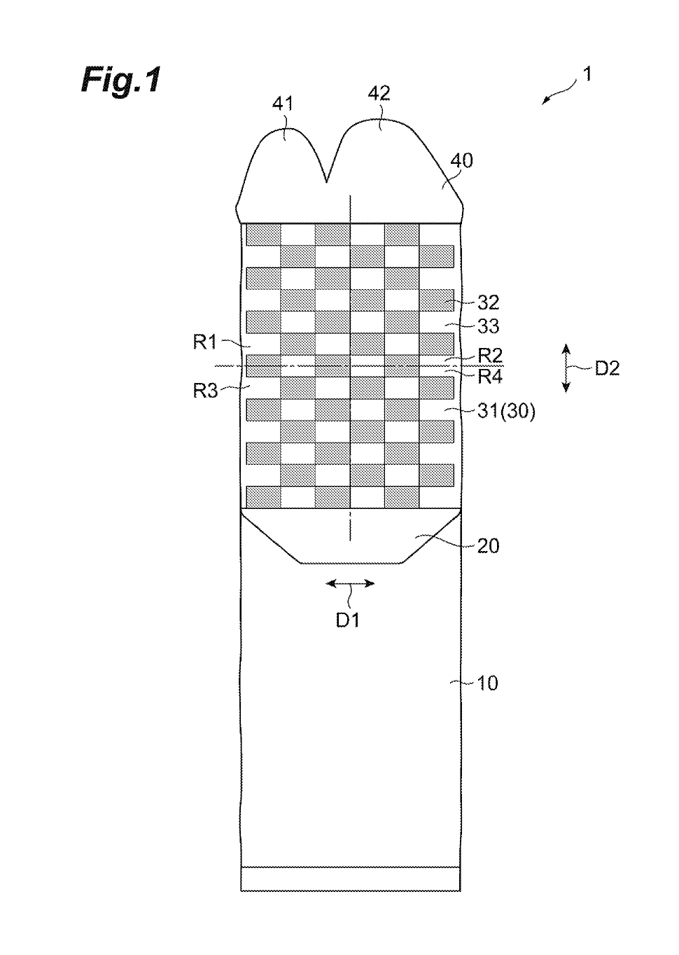

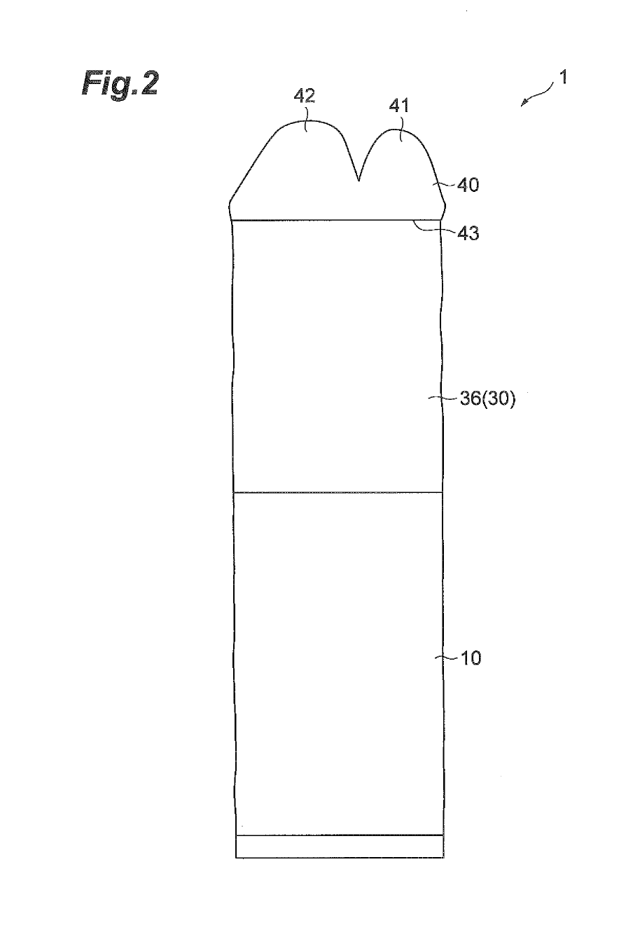

FIG. 1 is a sole side plan view of an inner side (back side, skin side) of a sole stimulation sock (for the right foot) according to the embodiment of the present invention in a state where the sole stimulation sock is folded in a planar shape. FIG. 2 is an instep side plan view of the inner side (back side, skin side) of the sole stimulation sock (for the right foot) according to the embodiment of the present invention in the state where the sole stimulation sock is folded in a planar shape.

A sole stimulation sock 1 in FIG. 1 and FIG. 2 is formed by sequentially knitting a cylindrical portion 10 which covers the upper portion of the ankle, a heel portion 20 which covers the heel, a cylindrical portion 30 which covers the sole and the instep, and a toe portion 40 which covers the toes. For example, these types of socks are knitted by a circular knitting machine.

The circular knitting machine uses a full computer-type knitting machine which is provided with a cylindrical cylinder. The full computer type indicates a knitting system in which stitch design data of the whole product which is created on a computer can be transferred to a knitting machine through an electromagnetic recording medium and automatic knitting can be electrically performed based on the stitch design data, and a series of work of designing and knitting is processed using the computer.

In this type of the circular knitting machine, a cylindrical cylinder rotates, knitting needles which are arranged on the circumference repeat vertical motion, and knitting yarn is knitted in a loop shape (plain knitting, pile knitting, mesh knitting, or the like). One course of knitting is performed by a rotation (one turn) of 360 degrees and cylindrical portions 10 and 30 are knitted by repetition of rotary knitting in the same direction. The counterclockwise rotation of the cylinder is generally called normal rotation and the clockwise rotation thereof is generally called reverse rotation.

In contrast, when knitting the heel portion 20 and the toe portion 40, the cylinder repeats the normal rotation and the clockwise rotation in a unit of one course and an arbitrary needle is used for knitting. Such a motion of the cylinder is generally called reciprocating rotation. When knitting half of the heel portion 20, needling up (or narrowing) is performed, and when knitting the remaining half of the heel portion 20, needling down (or widening) is performed. A grafting line between the needling up-knitting portion and the needling down-knitting portion of the heel portion 20 is a gore line. Similarly, when knitting half of the toe portion 40, needling up (or narrowing) is performed, and when knitting the remaining half of the toe portion 40, needling down (or widening) is performed. A grafting line between the needling up-knitting portion and the needling down-knitting portion of the toe portion 40 is a gore line. In addition, a grafting portion (seam allowance) 43 is sewn up in order to close the space between the toe portion 40 and the cylindrical portion 30.

The cylindrical portion 10 is for example, in a crew length. Accordingly, it is possible to secure heat retaining properties since the length is not too short, and the socks are easily worn and taken off since the length is not too long.

The inner side (back side, skin side) of the heel portion 20 is knitted through pile knitting. The whole portion of the inner side of the heel portion 20 may be knitted through the pile knitting or at least a grounding portion thereof may be knitted through the pile knitting. Accordingly, it is possible to protect the heel of a wearer from weighted stimulation during, for example, grounding (landing) in a walking motion.

The inner side (back side, skin side) of the toe portion 40 is knitted through pile knitting. The whole portion of the inner side of the toe portion 40 may be knitted through the pile knitting or at least a grounding portion thereof may be knitted through the pile knitting. Accordingly, it is possible to protect the toes of a wearer from weighted stimulation during, for example, kicking in a walking motion.

In addition, the toe portion 40 has a divided structure (bifurcated structure) which is divided into two regions including a region 41 that accommodates the big toe of a wearer and a region 42 which accommodates other toes. The region 42 which accommodates toes other than the big toe may be further divided into a plurality of regions. Accordingly, it is possible to suppress the socks from twisting while the socks are worn.

In addition, the grafting portion 43 between the toe portion 40 and the cylindrical portion 30 is knitted through a linking seam or a front rosso seam. Accordingly, it is possible to protect the toes of a wearer from stimulation due to the grafting portion 43 between the toe portion 40 and the cylindrical portion 30.

The cylindrical portion 30 is formed with a sole portion 31 which covers the sole of a wearer and an instep portion 36 which covers the instep of a wearer. The inner side (back side, skin side) of the sole portion 31 is formed with a projected portion 32 and a recessed portion 33. The projected portion 32 is knitted through pile knitting and the recessed portion 33 is knitted through mesh knitting. In addition, the instep portion 36 is also knitted through mesh knitting. The projected portion of the inner side (back side, skin side) of the sole portion 31 protrudes toward the skin side. Accordingly, the weighted stimulation during, for example, grounding (landing) in a walking motion is moderated, and therefore, it is possible to apply stimulation which is equivalent to manual mechanical stimulation which is applied by a health professional, to the sole. In a case where a projected portion and a recessed portion are provided on the outer side (front side) of the sole portion 31 and the projected portion protrudes toward the surface, there is no effect of applying weighted stimulation during grounding (landing) as stimulation which is equivalent to manual mechanical stimulation which is applied by a health professional, to the sole.

In the inner side of the sole portion 31, the proportion of the area A1 of all of the projected portions 32 is 26% to 68% with respect to the sum of the area A1 of all of the projected portions 32 and the area A2 of all of the recessed portions 33. in other words, with respect to 100% of the sum of the area A1 of all of the projected portions 32 and the area A2 of all of the recessed portions 33, that is, with respect to the area A1+A2=100% of the sole portion 31, the ratio of the area A1 of all of the projected portions 32 to the area A2 of all of the recessed portions 33 is A1:A2=26% to 68%: 74% to 32%. In the inner side of the sole portion 31, in a case where the proportion of the area A1 of all of the projected portions 32 is less than 26% with respect to the total of the area A1 of all of the projected portions 32 and the area A2 of all of the recessed portions 33, the number of projected portions 32 is small, and therefore, only a local region in the sole can be stimulated. Accordingly, it is impossible to apply stimulation which is equivalent to manual mechanical stimulation which is applied by a health professional, to the sole. In the inner side of the sole portion 31, in a case where the proportion of the area A1 of all of the projected portions 32 is greater than 68% with respect to the total of the area A1 of all of the projected portions 32 and the area A2 of all of the recessed portions 33, the stimulation effect decreases while it is possible to protect the sole of a wearer from weighted stimulation during, for example, grounding (landing) in a walking motion since the projected portions 32 come into contact with the sole in a wide range. Therefore, it is impossible to apply stimulation which is equivalent to manual mechanical stimulation which is applied by a health professional, to the sole.

In a case where the inner side of the sole portion 31 is divided into four division regions R1, R2, R3, and R4 by being equally divided into two regions in a right-left direction D1 and a toe-heel direction D2, the difference between the proportion of the area of all of projected portions 32 in any one division region of the four division regions and the proportion of the area of all of projected portions 32 in other division regions is less than or equal to 30%. Specifically, the difference between the proportion of the area of all of projected portions 32 in the division region R1 and the proportion of the area of all of projected portions 32 in the division regions R2, R3, and R4 is less than or equal to 30%; the difference between the proportion of the area of all of projected portions 32 in the division region R2 and the proportion of the area of all of projected portions 32 in the division regions R3, and R4 is less than or equal to 30%; and the difference between the proportion of the area of all of projected portions 32 in the division region R3 and the proportion of the area of all of projected portions 32 in the division region R4 is less than or equal to 30%. Accordingly, projected portions and recessed portions are substantially uniformly distributed over the entire sole portion 31, and therefore, it is possible to apply stimulation which is equivalent to manual mechanical stimulation which is applied by a health professional, to the sole. In a case where the difference between the proportion of the area of all of projected portions 32 in any one division region of the four division regions and the proportion of the area of all of projected portions 32 in other division regions exceeds 30%, deviation is caused in the distribution of the projected portions and the recessed portions in the sole portion 31, and therefore, it is impossible to apply stimulation which is equivalent to manual mechanical stimulation which is applied by a health professional, to the entire sole.

The size of each of the projected portions 32 may vary and the average value of the areas of the projected portions 32 is 7 mm² to 650 mm². If the area of each of the projected portions 32 is greater than or equal to 7 mm² (minimum value of the fingertip contact area when performing manual mechanical stimulation), it is possible to suppress damage of the delicate sole of a patient with diabetes or the like. In contrast, the area of each of the projected portions 32 is less than or equal to 650 mm² and is a size equivalent to the ball (maximum value of the contact area in the entire ball of the thumb when applying manual mechanical stimulation) of a finger. In a case where the average value of the areas of the projected portions 32 is less than 7 mm²; the areas of the projected portions 32 are extremely small; the projected portions 32 are hard; and the shapes of the projected portions 32 are stable, the pressure accompanied with weighted stimulation increases. Therefore, there is a concern that the delicate sole of a patient with diabetes may be damaged. In a case where the average value of the areas of the projected portions 32 is less than 7 mm²; the area of the projected portions 32 is extremely small; the projected portions 32 are soft; and the shapes of the projected portions 32 are unstable and easily deformed, the pressure accompanied with weighted stimulation does not increase, but is transferred to the sole without being moderated by the projected portions 32. Therefore, there is a concern that the delicate sole of a patient with diabetes may be damaged. In addition, in a case where the average value of the areas of the projected portions 32 exceeds 650 mm², the projected portions 32 come into contact with the sole in a wide range, and therefore, it is possible to protect the sole of a wearer from weighted stimulation during, for example, grounding (landing) in a walking motion. However, it is impossible to apply stimulation which is equivalent to manual mechanical stimulation which is applied by a health professional, to the sole due to a decreased stimulation effect.

In addition, the sizes of the projected portions 32 may be substantially the same as each other or the area of each of the projected portions 32 may be 7 mm² to 150 mm². The area of each of the projected portions 32 is less than or equal to 150 mm² and is a size equivalent to the ball (maximum value of the fingertip contact area when applying manual mechanical stimulation) of a finger.

In the mode shown in FIG. 1, the projected portion 32 and a recessed portion 33 commonly have a rectangular shape and have the same size (area) as each other. In addition, the projected portions 32 and the recessed portions 33 are two-dimensionally alternately arranged in a regular manner.

As the material of knitting yarn of the sole stimulation sock 1, yarn is preferably used which is excellent in moisture absorbing/releasing properties, water absorbing properties, and quick-drying properties. Examples thereof include wool, polyester with a modified cross section, and nylon “Quup” which is manufactured by Toray Industries, Inc. Accordingly, it is possible to obtain the sole stimulation sock which is excellent in moisture absorbing/releasing properties and in shape stability of a pile loop (being hard to lose its strength due to the risen pile) in a projected portion 32, and to secure softness equivalent to the ball (fingertip when performing manual mechanical stimulation) of a finger.

In addition, the knitting yarn of the sole stimulation sock 1 may have a white color or a comparatively light color. Accordingly, it is possible to recognize the state of the foot (for example, outflow of body fluids such as hemorrhaging, and the presence or absence of external injuries) from the external appearance even when the sole stimulation sock is worn.

In addition, processing of suppressing the growth of Trichophyton may be subjected to the knitting yarn of the sole stimulation sock 1 or the inner and outer surfaces (front and rear surfaces) of the sole stimulation sock 1. Accordingly, it is possible to suppress the development of tinea pedis.

As described above, according to the sole stimulation sock 1 of the present embodiment, the average value of the areas of the projected portions 32 in the inner side of the sole portion 31 is 7 mm² to 650 mm² which is a range between the size when the sole is lightly brought into contact with a fingertip and the size when the sole is brought into contact with the entire ball of a finger; the projected portions 32 have softness equivalent to the ball of a finger by being knitted through pile knitting; and the proportion of the area of all of the projected portions 32 is 26% to 68% with respect to the sum of the area of all of the projected portions 32 and the area of all of the recessed portions 33. Therefore, it is possible to reproduce stimulation which is equivalent to manual mechanical stimulation which is applied by a health professional. In addition, the difference between the proportion of the area of projected portions 32 in any one division region of the four division regions R1, R2, R3, and R4, which are obtained such that the inner side of the sole portion 31 is equally divided into two regions in a right-left direction D1 and a toe-heel direction D2, and the proportion of the area of projected portions 32 in other division regions is less than or equal to 30%. Therefore, the projected portions 32 are sparsely scattered in the inner side of the sole portion 31 without being deviated. Accordingly, it is possible to apply stimulation which is equivalent to manual mechanical stimulation which is applied by a health professional, to the sole by only being worn in a daily life (for example, by only walking) and to improve neuropathy in a foot portion.

In addition, according to the sole stimulation sock 1 of the present embodiment, it is possible to secure air permeability since the recessed portions 33 of the sole portion 31 and the instep portion 36 are knitted through mesh knitting. Accordingly, it is possible to suppress the growth of bacteria caused by stuffiness and to prevent the development of infectious disease of a foot portion.

In addition, according to the sole stimulation sock 1 of the present embodiment, it is possible to expect an effect of improving neuropathy in a foot portion for all people who have neuropathy in their feet due to certain causes without being limited to neuropathy of patients with diabetes.

In addition, according to the sole stimulation sock 1 of the present embodiment, it is possible to expect acceleration of the circulation of blood in a foot portion by applying stimulation which is equivalent to manual mechanical stimulation which is applied by a health professional, to the sole, and therefore, it is possible to expect improvement in a circulatory disorder in the foot portion. Furthermore, according to the sole stimulation sock 1 of the present embodiment, it is possible to expect acceleration of circulation of blood in the foot portion of a healthy person and improvement in perception sensitivity of the sole.

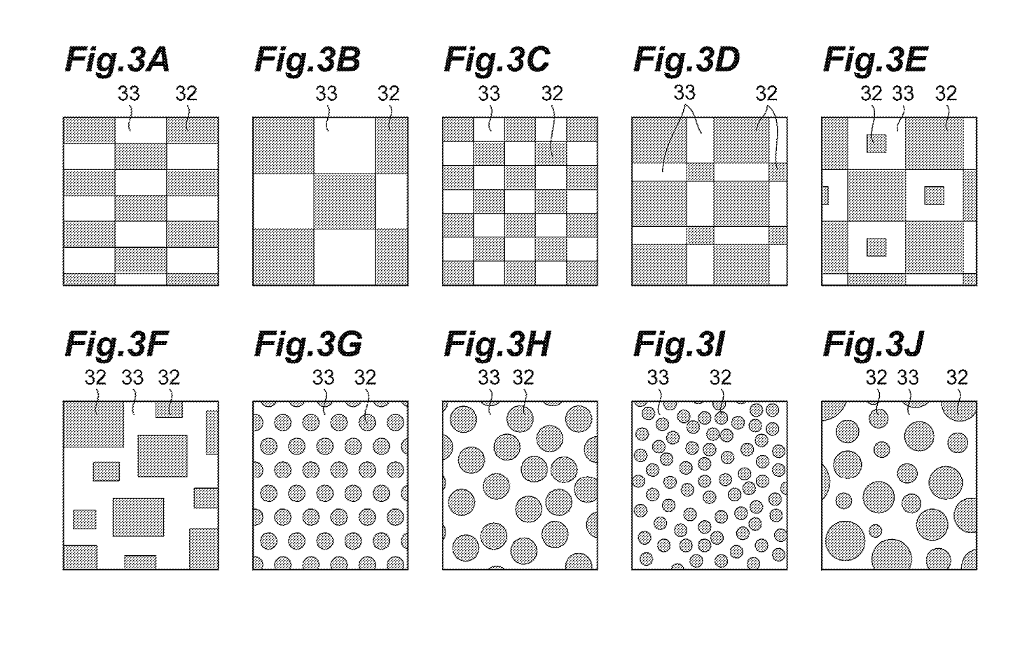

The present invention is not limited to the above-described present embodiment and various modifications can be made. In the present embodiment, the mode in which the projected portions 32 and the recessed portions 33 commonly have a rectangular shape and are same size (area) as each other, and are two-dimensionally arranged in an alternating regular manner has been exemplified (FIG. 3A). However, the projected portions 32 and the recessed portions 33 are not limited to the present embodiment.

For example, as shown in FIGS. 3B and 3C, the projected portions 32 and the recessed portions 33 may have a square shape instead of the rectangular shape. In addition, as shown in FIGS. 3D and 3E, two kinds or more of projected portions 32 which are different sizes than each other may be included as the projected portions 32. In FIG. 3D, two kinds of the projected portions 32 which are different sizes than each other are alternately arranged in a regular manner while the projected portions 32 and the recessed portions 33 are two-dimensionally arranged in an alternating regular manner. Moreover, the recessed portions 33 have a rectangular shape while the projected portions 32 have a square shape. In contrast, in FIG. 3E, among two types of projected portions 32 which are different sizes than each other, large projected portions and the recessed portions 33 are two-dimensionally arranged in an alternating regular manner and small projected portions 32 are arranged in the recessed portions 33. In addition, as shown in FIG. 3F, the projected portions 32 may be two-dimensionally arranged in an irregular manner through the recessed portions 33. In FIG. 3F, two kinds of projected portions 32 which are different sizes are irregularly arranged through the recessed portions 33.

In addition, as shown in FIG. 3G, the projected portions 32 may have a circular shape and be two dimensionally arranged in a regular manner at equal intervals through the recessed portions 33. In addition, as shown in FIGS. 3H and 31, the projected portions 32 may be two dimensionally arranged in an irregular manner through the recessed portions 33. In addition, as shown in FIG. 3J, two kinds or more of projected portions 32 which are different sizes than each other may be included as the projected portions 32. In FIG. 3J, two kinds of the projected portions 32 which are different sizes than each other are irregularly arranged through the recessed portions 33, but two kinds of the projected portions 32 may be regularly arranged in an alternating manner at equal intervals through the recessed portions 33. Although various modes of the projected portions 32 and the recessed portions 33 have been exemplified in FIGS. 3A to 3J, it is important that the projected portions 32 are sparsely scattered through the recessed portions 33 without being deviated, in order to stimulate the sole.

In addition, in the present embodiment, the mode has been exemplified in which the projected portions 32 are knitted through pile knitting, but the projected portions 32 are not limited thereto. For example, the projected portions 32 may be formed by a projection through multiple tuck knitting or may be formed by switching the material through a cut-both method.

In addition, the projected portions 32 may be formed of a resin, a synthetic resin, or synthetic rubber. In this case, examples of the material of the projected portions 32 include polyurethane, silicone, polyvinyl chloride, and synthetic rubber. If stimulation applied to a patient with diabetes is too strong, subcutaneous hemorrhaging is caused, thereby causing a lesion in the foot. However, according to the invention, it is possible to reproduce softness equivalent to the ball (fingertip when performing manual mechanical stimulation) of a finger and to apply stimulation which is equivalent to manual mechanical stimulation which is applied by a health professional, to the sole.

In addition, the projected portions 32 may be formed such that a separate member such as a patch cloth which is formed by pile knitting, a projection through multiple tuck knitting, or switching the material through a cut-both method, or a separate member which is formed of a resin, a synthetic resin, or synthetic rubber is adhered to a sole portion in a cylindrical portion.

### EXAMPLE

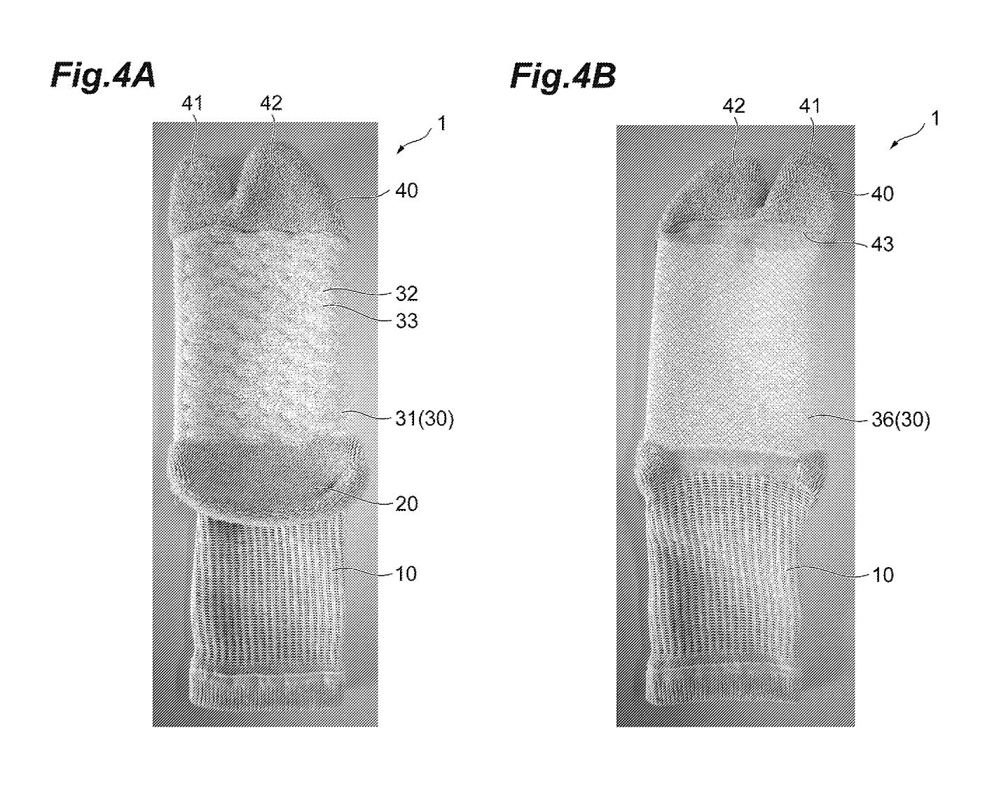

The sole stimulation sock 1 of the present embodiment shown in FIGS. 1 and 2 was produced as Example 1 shown in FIGS. 4A and 4B to perform the following evaluation.

In Example 1, one piece of No. 48 count two-folded yarn of wool and one piece of No. 70 denier two-folded yarn of wooly nylon were used to knit front yarn of the cylindrical portion 30; two pieces of No. 32 single yarn of cotton acrylic blended yarn was used to knit front yarn of the toe portion 40 and the heel portion 20; two pieces of No. 30 single yarn of polyester cotton blended yarn was used to knit front yarn of the cylindrical portion 10, and yarn, which covered No. 70 denier nylon yarn as No. 30 denier polyurethane core yarn, was used to knit back yarn. In the inner side of the sole portion 31, the proportion of the area of all of the projected portions 32 was set to 50% with respect to the sum of the area of all of the projected portions 32 and the area of all of the recessed portions 33, and the difference between the proportion of the area of projected portions 32 in any one division region of the four division regions R1, R2, R3, and R4 in the inner side of the sole portion 31 and the proportion of the area of projected portions 32 in other division regions was set to 0%. The area of each of the projected portions 32 was set to 15 mm×10 mm=150 mm². The thickness of a projected portion 32, that is, the thickness of a pile was set to 3 mm. The toe portion 40 and the heel portion 20 has high cushioning properties compared to the cylindrical portion 30 and the sole portion 31, and therefore, a more smooth material was used for the toe portion and the heel portion.

### [Evaluation 1]

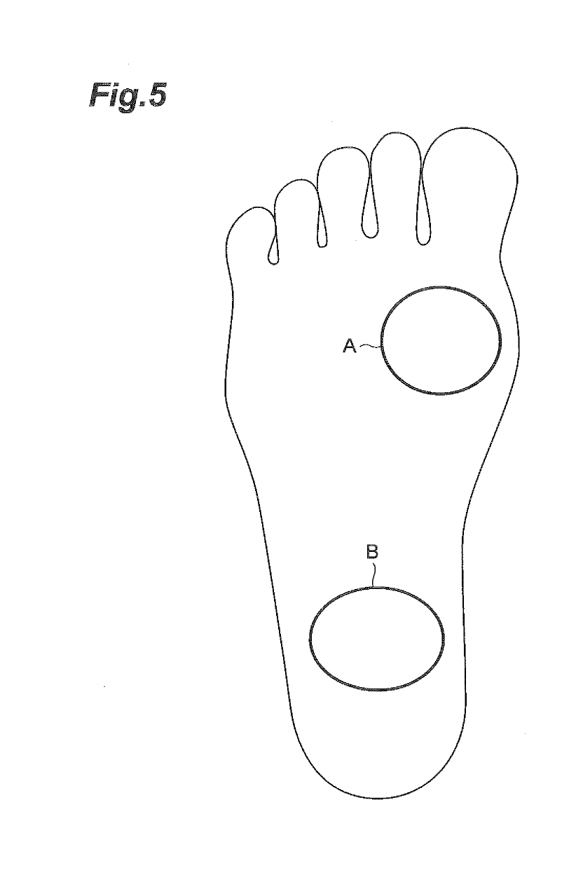

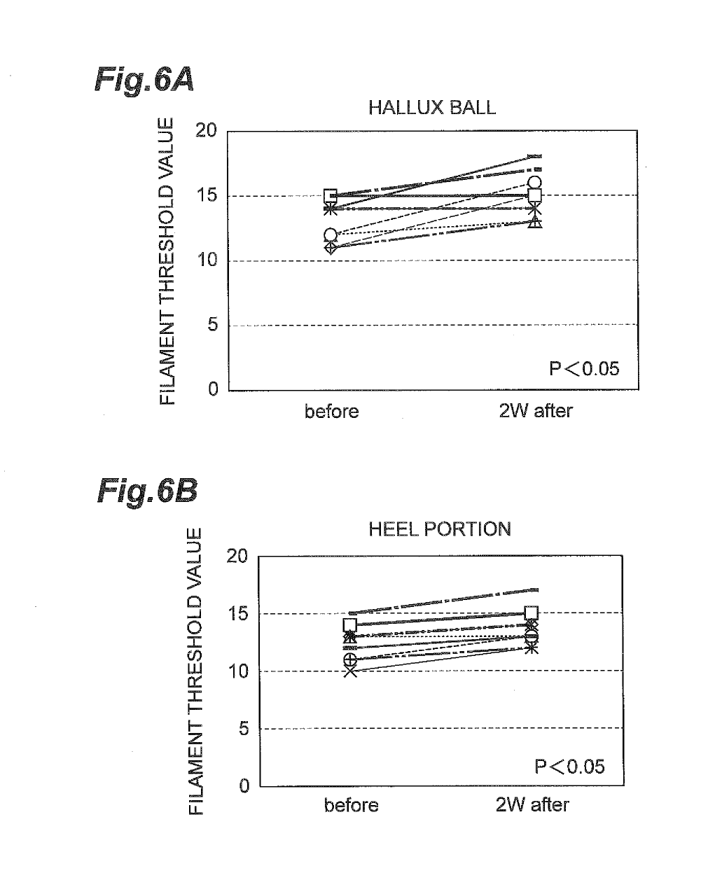

In Evaluation 1, 9 examinees were allowed to wear the socks in Example 1 for two weeks, and a perception examination of the sole before wearing the socks and 2 weeks after wearing the socks was performed. The age of the 9 examinees was 71±11 years old, the ratio of males and females thereof was 5:4; their disease duration of diabetes was about 20 years; and they had neuropathy. In the perception examination, a monofilament was brought into contact with the hallux ball A of a sole portion and the heel portion B (FIG. 5) of each examinee using a monofilament perception tester (Semmes-Weinstein Monofilaments). The monofilament which was to be brought into contact with the hallux ball and the heel portion was changed from a thin filament to a thick filament in order to examine the thickness at which the examinees can feel. The results of the perception examination are shown in FIGS. 6A and 6B.

FIG. 6A shows filament threshold values in the hallux balls A of the 9 examinees and FIG. 6B shows filament threshold values in the heel portions B of the 9 examinees. A threshold value indicates a thickness at which an examinee can feel stimulation using filaments which differ in thickness. It was determined whether or not there is an improvement in the perception at a significant level of 5%, using a Wilcoxon signed-rank test with reference to FIGS. 6A and 6B. According to FIGS. 6A and 6B, the critical rate P is lower than 5% of the significant level, in other words, the variation in the degree of the improvement in the perception of the 9 examinees is small, and therefore, it can be determined that there is an effect of improving the perception with a significant difference.

### [Evaluation 2]

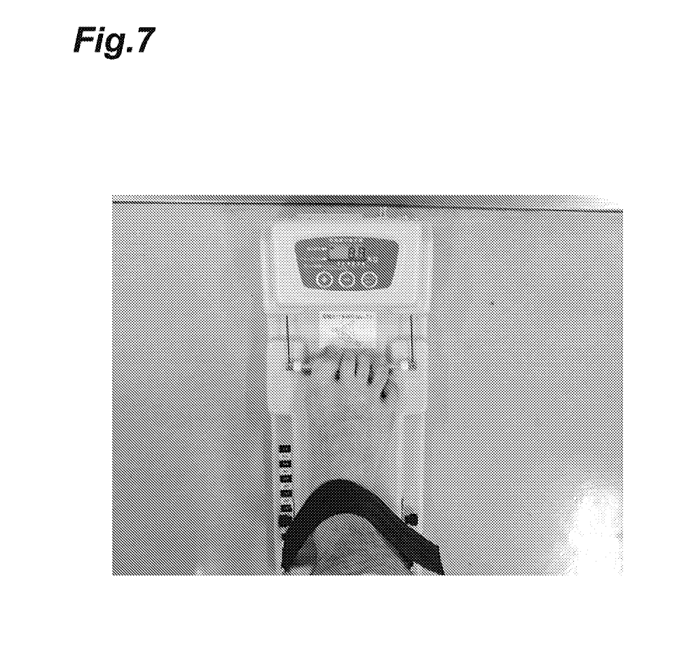

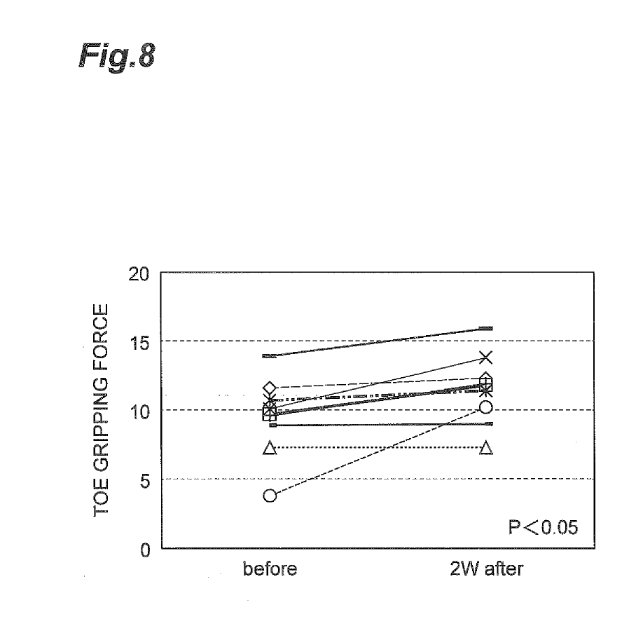

In Evaluation 2, the above-described 9 examinees were allowed to wear the socks in Example 1 for two weeks, and a toe gripping force examination before wearing the socks and 2 weeks after wearing the socks was performed. In the toe gripping force examination, as shown in FIG. 7, the toe gripping force was measured by controlling second to fourth toes so as to hang over a toe gripping bar around the distal phalanx of the hallux. The results of the toe gripping force examination are shown in FIG. 8.

FIG. 8 shows toe gripping force for the 9 examinees. It was determined whether or not there was an effect of improving the toe gripping force with a significant difference of 5% using a paired t-test with reference to FIG. 8. According to FIG. 8, the critical rate P is lower than 5% of the significant level, in other words, the variation in the degree of the improvement in the toe gripping force of the 9 examinees is small, and therefore, it can be determined that there is an effect of improving the toe gripping force with a significant difference.

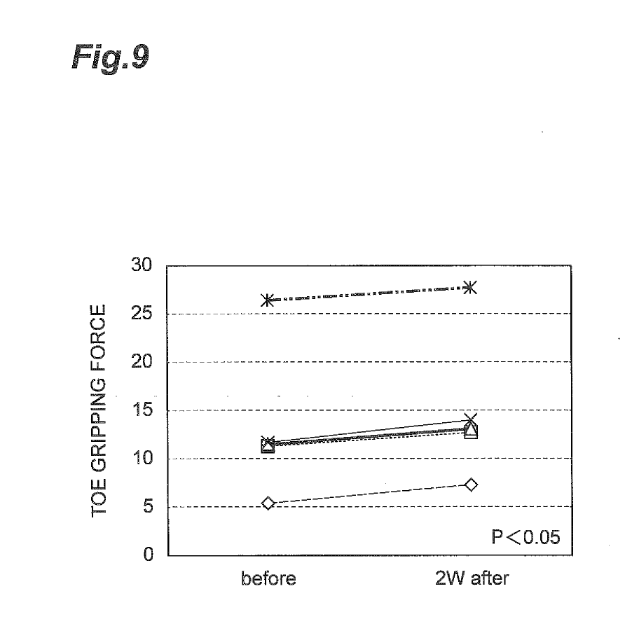

Here, according to Example 1, it was worthy of special mention that it was found that there was also an improvement effect in a patient with diabetes who does not have neuropathy. FIG. 9 shows results in which the similar toe gripping force examination described above was performed on 5 examinees who do not have neuropathy. The age of the 5 examinees was 67±17 years old, the ratio of males and females thereof was 2:3; their disease duration of diabetes was about 13 years; and they did not have neuropathy. According to FIG. 9, the critical rate P is lower than 5% of the significant level, in other words, the variation in the degree of the improvement in the toe gripping force of the 5 examinees is small, and therefore, it can be determined that there is an effect of improving the toe gripping force with a significant difference.

### [Consideration]

For example, it is said that the disorder which is the most frequently developed is diabetic neuropathy among complications of diabetes and fine capillaries becomes weak when diabetic neuropathy is developed. When the capillary is damaged which carries blood to a peripheral nerve, it is impossible to carry sufficient oxygen and nutrition, thereby negatively affecting a nerve which controls a sense or a motion, and an autonomic nerve. In this manner, a patient with diabetes has decreased perception due to neuropathy, and therefore, is easily wounded without noticing shock or heat. As a result, the risk of causing a lesion in the foot increases. Furthermore, it is difficult to heal a wound, which is comparatively easily cured in a case of a healthy person, of a patient with diabetes whose blood flow is poor. In addition, the patient with diabetes has decreased toe gripping force, and therefore, there is a high risk of falling down. In addition, bacteria may grow since the patient with diabetes does not notice stuffiness, and as a result, the development risk of infectious disease is increased.

Furthermore, when neuropathy progresses, an ulcer in terminal tissues starts to be generated, and gangrene and necrosis start. It is said that diabetic neuropathy easily occurs in the foot of the body. When gangrene and necrosis progress, in the worst case, a foot portion needs to be amputated. It is said that the number of patients whose foot portion is amputated due to complications from diabetes is 3,000 per year.

When a health professional applies manual mechanical stimulation to a foot portion which is a terminal tissue, repeated weak stimulation which is applied to subcutaneous tissue increases stimulation to the central nervous system through mechanoreceptors, Merkel's discs, and Ruffini-like endings. It is considered that this leads to activation of the movement of a nerve tissue itself which is decreased by being induced by diabetes, and prevention and recovery of decline of a sensory nerve. Accordingly, there is also a favorable effect on blood vessels or muscles, and improvement in blood flow, coldness, and the like is expected.

The manual mechanical stimulation can be received from a health professional by going to the hospital, but is also desirably applied to the feet by himself/herself daily. However, applying the manual mechanical stimulation to the feet by himself/herself everyday requires a lot of time.

In regard to these points, when referring to the results of Evaluations 1 and 2 according to the present Example 1, it can be seen that according to the sole stimulation sock 1 of the present Example 1, it is possible to apply stimulation which is equivalent to manual mechanical stimulation which is applied by a health professional, to blood vessels and nerves of a foot portion by only being worn in daily life (for example, by only walking), and to thereby obtain an effect of promoting the blood flow in the foot portion, an effect of maintaining and improving perception of terminal nerves of the foot portion (effect of improving neuropathy), and an effect of maintaining and improving toe gripping force (effect of improving neuropathy). Furthermore, according to the sole stimulation sock 1 of the present Example 1, it is possible to perform prevention of a lesion in the foot from being generated and early detection due to improvement in the perception, and prevention of falling down due to improvement in the toe gripping force.

What is claimed is:

1. A sole stimulation sock,
wherein an inner side of a sole portion in a cylindrical portion between a heel portion which covers the heel and a toe portion which covers the toes is formed with projected portions and recessed portions,
wherein in the inner side of the sole portion, a proportion of an area of all of the projected portions, with respect to the sum of the area of all of the projected portions and an area of all of the recessed portions, is 26% to 68%,
wherein the difference between a proportion of an area of the projected portions in any one division region of four division regions and a proportion of an area of the projected portions in other division regions is less than or equal to 30%, the four division regions being obtained such that the inner side of the sole portion is equally divided into two regions in a right-left direction and a toe-heel direction,
wherein the average value of the areas of the projected portions is 7 mm² to 650 mm², and
wherein the recessed portions are knitted by mesh knitting.

2. The sole stimulation sock according to claim 1,
wherein the projected portions are knitted by pile knitting.

3. The sole stimulation sock according to claim 1,
wherein the projected portions are formed of a resin, a synthetic resin, or synthetic rubber.

4. The sole stimulation sock according to claim 1,
wherein an instep portion in the cylindrical portion is knitted by mesh knitting.

5. The sole stimulation sock according to claim 1,
wherein the inner side of the heel portion is knitted by pile knitting.

6. The sole stimulation sock according to claim 1,
wherein an inner side of the toe portion is knitted by pile knitting.

7. The sole stimulation sock according to claim 1,
wherein the toe portion has a divided structure which is divided into two or more regions which accommodate the toes of a wearer.

8. The sole stimulation sock according to claim 1,
wherein a grafting portion between the toe portion and the cylindrical portion is knitted by a linking seam or a front rosso seam.

9. The sole stimulation sock according to claim 1,
wherein processing of suppressing the growth of Trichophyton is performed.
